(19) United States
(12) Patent Application Publication
(10) Pub. No.: US 2025/0001076 A1
(43) Pub. Date: Jan. 2, 2025

(54) Screen Control Method for Infusion Pump, Apparatus, Infusion Pump, Device and Medium

(71) Applicant: Medcaptain Medical Technology Co., Ltd., Shenzhen (CN)

(72) Inventors: Shengping LI, Shenzhen (CN); Ping LI, Shenzhen (CN); Wenhui YANG, Shenzhen (CN)

(73) Assignee: Medcaptain Medical Technology Co., Ltd., Shenzhen (CN)

(21) Appl. No.: 18/752,910

(22) Filed: Jun. 25, 2024

(30) Foreign Application Priority Data
Jun. 30, 2023 (CN) 202310801319.3

(51) Int. Cl. A61M 5/172

(52) U.S. Cl. CPC A61M 5/1723

(57) ABSTRACT

This invention provides a screen-based method for controlling an infusion pump, an apparatus, an infusion pump, a device, and a medium. The method includes: generating an information display interface according to an operating state of the infusion pump; displaying the information display interface by a touch screen of the infusion pump; and stopping processing of a manipulation command received by the touch screen in response to an opening operation of the pump door of the infusion pump. The invention can solve the problem in the prior art that it is easy to mistakenly touch the display content on the touch screen of the infusion pump, which leads to low efficiency of the infusion operation and affects the effect of the infusion. The invention thereby improves the safety and reliability of the infusion pump.

### REFERENCE TO RELATED APPLICATIONS

The invention claims priority of Chinese Patent Application No. 202310801319.3, entitled “Screen Control Method for Infusion Pump, Apparatus, Infusion Pump, Device and Medium” filed with the China National Intellectual Property Administration on Jun. 30, 2023, which is incorporated herein by reference in its entirety.

### TECHNICAL FIELD

This invention generally relates to the field of intelligent medical technologies, and more particularly, to a screen control method for an infusion pump, apparatus, infusion pump, device and storage medium.

### BACKGROUND ART

In clinical treatment scenarios involving intravenous infusion, infusion pumps, as intelligent infusion devices, have been widely adopted in the healthcare industry, largely replacing conventional infusion sets.

However, during the process of performing infusion operations with the infusion pump, it is sometime easy to mistakenly touch the screen interface of the infusion pump and change the parameters displayed or set on the touch screen. Such parameters may include, for example, those relating to installing of consumables on the infusion pump when the infusion pump has finished setting the parameters; to changing the medication on the infusion pump when the infusion is suspended due to the change of the physiological parameters of the target patient; to eliminating the bubbles in the infusion line or the detaching the infusion line in the process of the infusion.

In all of the above cases, it is necessary to open or close the pump door of the infusion pump to install or replace the consumables, and it is easy to mistakenly touch the screen interface of the infusion pump and affect the parameters displayed or set on the touch screen when the user manually operates the pump door and when starting or exiting the infusion. In this circumstance, the operating user is required to reset the parameters or re-infuse the patient, which is inefficient and affects the therapeutic effect. Alternatively, if the user does not realize that the set parameters have been modified in time and continues the infusion, it may lead to medical errors.

### SUMMARY OF THE INVENTION

This invention provides a screen control method for an infusion pump, apparatus, device, and medium, for solving the problem of easily mistakenly touching the display content on the touch screen of the infusion pump in the prior art, and which can realize the technical effect of improving the safety and reliability of the infusion pump.

On the one hand, this invention provides a screen control method for an infusion pump, the method comprising:
generating an information display interface according to an operating state of the infusion pump;
displaying the information display interface by a touch screen of the infusion pump; and
stopping processing of a manipulation command (that is, a user-entered pump control command) received by the touch screen in response to an opening operation of the pump door of the infusion pump.

In an embodiment, the method further comprises:
resuming processing of the manipulation command received by the touch screen in response to receiving an unlocking command.

In an embodiment, the method further comprises:
stopping processing of a manipulation command received by a physical button provided on the infusion pump while stopping processing of the manipulation command received by the touch screen; and/or resuming processing of the manipulation command received by the physical button in response to receiving an unlocking command.

In an embodiment, the step of stopping processing of the manipulation command received by the touch screen comprises:
triggering the stopping of processing of the manipulation command received by the touch screen according to a set locking condition.

In an embodiment, the method further comprise:
acquiring unlocking operation data of an unlocking operation performed by an operating user of the infusion pump; and
adjusting the locking condition according to the unlocking operation data.

In an embodiment, the method further comprises:
acquiring biometric data of an operating user of the infusion pump;
determining user privileges authorized for the operating user; and
determining whether to generate the unlocking command according to the biometric data and the user privileges.

In addition, this invention provides an infusion pump, comprising a drive device, a main body housing, a pump door, a touch screen, a pump door detecting device, a storage device, and a controller;

wherein the pump door is movably connected to the main body housing; the drive device, the pump door detecting device, the storage device, and the controller are provided within the main body housing; the touch screen is provided on a side of the pump door away from the main body housing; the pump door detecting device is configured to detect an opening state of the pump door, and the controller is configured to control the drive device to push out the liquid in an infusion consumable connected to the infusion pump;

the controller is further configured to:

generate an information display interface according to an operating state of the infusion pump;

display the information display interface on a touch screen of the infusion pump; and

stop processing of a manipulation command received by the touch screen in response to an opening operation of the pump door of the infusion pump.

In an embodiment, the controller is further configured to resume processing of the manipulation command received by the touch screen in response to receiving an unlocking command.

In an embodiment, the infusion pump further comprises:
a physical button, the physical button being provided on the main body housing and connected to the controller; wherein the controller is further configured to stop processing of a manipulation command received by the physical button provided on the infusion pump while stopping processing of the manipulation command received by the touch screen; and/or resume processing of the manipulation command received by the physical button in response to receiving an unlocking command.

In an embodiment, the controller is further configured to trigger the stopping of processing of the manipulation command received by the touch screen according to a set locking condition.

The invention also provides a screen control apparatus for an infusion pump, comprising:
a generation module configured to generate an information display interface according to an operating state of the infusion pump;
a display module configured to display the information display interface by a touch screen of the infusion pump; and
a first control module configured to stop processing of a manipulation command received by the touch screen in response to an opening operation of the pump door of the infusion pump.

In an embodiment, the apparatus further comprises:
a second control module configured to resume processing of the manipulation command received by the touch screen in response to receiving an unlocking command.

In an embodiment, the apparatus further comprises:
a third control module configured to stop processing of a manipulation command received by a physical button provided on the infusion pump while stopping processing of the manipulation command received by the touch screen; and
a fourth control module configured to resume processing of the manipulation command received by the physical button in response to receiving an unlocking command.

In an embodiment, the first control module is further configured to trigger the stopping of processing of the manipulation command received by the touch screen according to a set locking condition.

In an embodiment, the apparatus further comprises:
a first acquisition module configured to acquire unlocking operation data of an unlocking operation performed by an operating user of the infusion pump; and
an adjustment module configured to adjust the locking condition according to the unlocking operation data.

In an embodiment, the apparatus further comprises:
a second acquisition module configured to acquire biometric data of an operating user of the infusion pump;
a first determining module configured to determine user privileges authorized for the operating user; and
a second determining module configured to determine whether to generate the above unlocking command according to the above biometric data and the above user privilege.

Furthermore, this invention provides an electronic device comprising: a processor, and a memory connected to the processor; wherein the memory stores computer-executable instructions; and the processor execute the computer-executable instructions stored in the memory to:
generate an information display interface according to an operating state of the infusion pump;
display the information display interface using a touch screen of the infusion pump; and
stop processing of a manipulation command received by the touch screen in response to an opening operation of the pump door of the infusion pump.

In an embodiment, the processor is further configured to resume processing of the manipulation command received by the touch screen in response to receiving an unlocking command.

In an embodiment, the processor is further configured to stop processing of a manipulation command received by a physical button provided on the infusion pump while stopping processing of the manipulation command received by the touch screen; and/or resume processing of the manipulation command received by the physical button in response to receiving an unlocking command.

In an embodiment, the processor is further configured to trigger the stopping of processing of the manipulation command received by the touch screen according to a set locking condition.

In an embodiment, after the step of stopping processing of the manipulation command received by the touch screen, the processor is further configured to:
acquire unlocking operation data of an unlocking operation performed by an operating user of the infusion pump; and
adjust the locking condition according to the unlocking operation data.

In an embodiment, the processor is further configured to:
acquire biometric data of an operating user of the infusion pump;
determine user privileges authorized for the operating user; and
determine whether to generate the unlocking command according to the biometric data and the user privileges.

Additionally, this invention provides a computer-readable storage medium, wherein the computer-readable storage medium stores computer-executable instructions, which are configured to realize a method according to any of the foregoing when executed by the processor.

This invention also provides a computer program product comprising a computer program, wherein the computer program implements a method as any of the foregoing when executed by the processor.

This invention provides a screen control method for an infusion pump, apparatus, infusion pump, device and medium, in which an information display interface is generated according to an operating state of the infusion pump; the information display interface is displayed by a touch screen of the infusion pump; and the processing of a manipulation command received by the touch screen is stopped in response to an opening operation of the pump door of the infusion pump.

In the screen control method provided by the embodiments of this invention, the processing of a manipulation command received by the touch screen is stopped in response to an opening operation of the pump door of the infusion pump, which can be understood as controlling the touch screen of the infusion pump to enter a lock screen protection state, so as to prevent incorrect operation from interfering with the normal work of the infusion pump. When the user performs operations such as installing or replacing consumables that require opening the pump door, accidental touches on the displaying or setting parameters on the touch screen of the infusion pump can thus be avoided. This eliminates the need for the user to reset or modify parameters, thus maintaining operational efficiency. Additionally, it prevents medical accidents, enhancing the effectiveness of infusion therapy administered to the patient using the infusion pump.

### BRIEF DESCRIPTION OF THE DRAWINGS

The accompanying drawings herein, which are incorporated into and form a part of the specification, illustrate embodiments consistent with this invention and are used in conjunction with the specification to explain the principles of this invention.

FIG. 1 is a schematic diagram of an architecture of an infusion pump provided by embodiments of this invention.

FIG. 2 is a flowchart of a screen control method of an infusion pump provided by embodiments of this invention.

FIG. 3 is a flowchart of a different screen control method of an infusion pump provided by embodiments of this invention.

FIG. 4 is a flowchart of yet another screen control method of an infusion pump provided by embodiments of this invention.

FIG. 5 is a flowchart of still another screen control method of an infusion pump provided by embodiments of this invention.

FIG. 6 is a structural block diagram of a screen control apparatus of an infusion pump provided by embodiments of this invention.

FIG. 7 shows a structural schematic diagram of an electronic device provided by embodiments of this invention.

By means of the above accompanying drawings, specific embodiments of this invention are shown, which will be described in greater detail later. These accompanying drawings and textual descriptions are not intended to limit the scope of this invention idea in any way, but rather to illustrate the concepts of this invention for those skilled in the art by reference to particular embodiments.

### DETAILED DESCRIPTION

Example embodiments will be described herein in detail, which are represented in the accompanying drawings. When the following description relates to the accompanying drawings, the same numerals in the different accompanying drawings indicate the same or similar elements unless otherwise indicated. The embodiments described in the following example embodiments do not represent all embodiments consistent with this invention. Rather, they are only examples of apparatuses and methods consistent with some aspects of this invention as detailed in the appended claims.

The terms covered by this invention are first explained:

Infusion pump: a medical instrument capable of accurately controlling the number of drops or the flow rate of an infused medication to ensure that the medication can be administered at a uniform rate, in an accurate amount, and safely enter the body of the patient in order to perform its function.

At present, intravenous infusion is a common way of drug delivery in clinical treatment. According to the nature of the drug, delivery of the drug will depend on the different physical characteristics of the different patients. For example, proper intravenous infusion speed may differ—infusion that is too fast or too slow may make it difficult to achieve the expected therapeutic effect, and even affect the safety of nursing. Therefore, infusion pumps, implemented as intelligent infusion devices with features to improve accuracy, safety and ease of use, have been widely used by the healthcare industry to replace ordinary infusion sets in a wide range of clinical settings.

However, in the clinical infusion scenario, during the process of performing infusion operations with the infusion pump, the following procedures may be necessary: installing consumables on the infusion pump when the infusion pump has finished setting the parameters; changing the medication on the infusion pump when the infusion is suspended due to the change of the physiological parameters of the target patient; to eliminating bubbles in the infusion line or the detaching the infusion line in the process of the infusion.

In all of the above cases, it is necessary to open or close the pump door of the infusion pump, for example, to install or replace the consumables, and it is easy to mistakenly touch the parameters displayed or set on the touch screen interface of the infusion pump when the user manually operates the pump door and when starting or exiting the infusion. In this circumstance, the operating user is required to reset the parameters or re-restart the infusion operation, which is inefficient and affects the therapeutic effect. Alternatively, if the user does not realize that the set parameters have been modified in time and continues the infusion, it may lead to medical errors.

This invention provides an infusion pump, and a screen control method of the infusion pump, which are intended to solve the technical problems of the prior art as described above.

The inventor of this invention, by considering that the touch screen of the infusion pump may be mistakenly touched, for example, when the pump door of the infusion pump is opened, the controller in the infusion pump stops processing of the manipulation command received by the touch screen of the infusion pump, that is, which can be understood as controlling the touch screen of the infusion pump to enter a lock screen protection state, so as to avoid mistakenly touching the information displayed on the touch screen of the infusion pump when the operating user manually operates the pump door. Afterwards, in response to the unlocking command received by the touch screen, the processing of the manipulation commands received by the touch screen described above is resumed, i.e., the touch screen is controlled to enter the unlocked display state, and the touch screen can receive the manipulation commands of the operating user normally.

In this way, when the operating user installs or replaces the consumables, or when starting or exiting the infusion, it is possible to avoid mistakenly touching the parameters displayed or set on the touch screen, and it is not necessary for the operating user to perform the parameter setting or modification operation again. Therefore, the operating efficiency of the operating user in using the infusion pump will not be affected, and medical accidents will not be caused, thereby improving the effect of the infusion pump being used to perform an infusion treatment on the execution subject.

The technical solution of this invention and how the technical solution of this invention solves the above-mentioned technical problems are described in detail with reference to the following specific embodiments. The following specific embodiments may be combined with each other, and the same or similar concepts or processes may not be repeated in some embodiments. Embodiments of this invention will be described below in conjunction with the accompanying drawings.

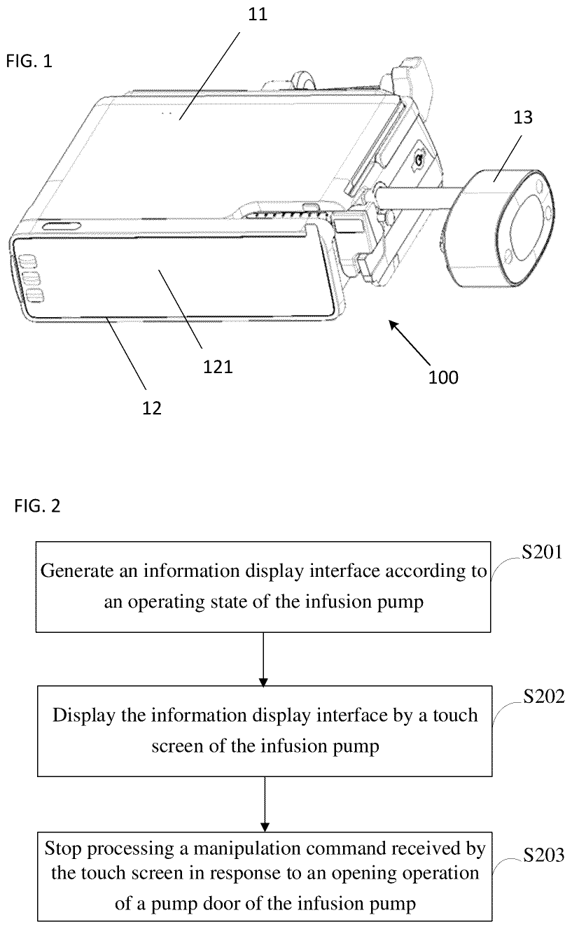

An embodiment of this invention provides an infusion pump, and FIG. 1 shows a schematic diagram of an architecture of an infusion pump provided by an embodiment of this invention. As shown in FIG. 1, the above-mentioned infusion pump 100 includes a main body housing 11, a pump door 12, a touch screen 121, a drive device, a pump door detecting device, a storage device, and a controller. The drive device, the pump door detecting device, the storage device, and the controller are provided in the above-mentioned main body housing 11, which is not shown in FIG. 1.

The above-mentioned pump door 12 is movably connected to the main body housing 11 (the pump door 12 can be opened and closed). The drive device, the storage device, and the controller are provided within the main body housing 11. The touch screen 121 is set on a side of the pump door 12 away from the main body housing 11. The pump door detecting device is configured to detect the opening state (may include the opening angle or degree) of the pump door 12, and may be set in or close to a side of the main body housing 11 facing the pump door 12, or set in or close to a side of the pump door 12 facing the main body housing 11, or distributed in the main body housing 11 or the pump door 12. The controller is configured to control the drive device to push out the liquid in the infusion consumable connected to the above infusion pump (pushed out by a push head assembly 13 shown in FIG. 1).

In the embodiment of this invention, the controller performs the following steps:
generating an information display interface according to the operating state of the infusion pump;
displaying the information display interface by a touch screen of the infusion pump;
in a first screen control mode, in response to an opening operation of the pump door of the infusion pump, stopping processing of a manipulation command received by the touch screen; and
in a second screen control mode, in response to a closing operation of the pump door of the infusion pump, stopping processing of the manipulation command received by the touch screen.

Specific application scenarios of this invention may be, but are not limited to, clinical medical or healthcare scenarios in which an infusion pump is used. The infusion pump is an intelligent injection device that pushes out the liquid in the infusion consumable connected to the infusion pump by a drive device, and is capable of accurately, uniformly, and continuously inputting medication into the body of the patient, in order to realize the infusion treatment of the patient.

Using the infusion pump 100 not only allows for strict control of medication dosage, ensuring the optimal effective concentration of the medication, and reasonably adjusting the injection speed, but also enables the continuous infusion of various urgently needed medications, reducing the occurrence of complications. Moreover, the infusion pump is easy to operate, provides precise and reliable infusion, and can effectively reduce the workload of clinical nursing.

In embodiments of this invention, by generating and displaying a corresponding information display interface according to the operating state of the infusion pump 100 on the touch screen 121 provided on the infusion pump 100, it is convenient for an operating user to understand and operate the infusion pump 100.

As an embodiment, for example, when the infusion pump 100 is in a standby state, the information display interface can display basic information such as the model number, power, time of the infusion pump 100. When the infusion pump 100 is in a working state, the information display interface can display important information such as the type of fluid to be infused, the flow rate, and the remaining volume. When the infusion pump 100 is in an alarm state, the information display interface can display prompt information such as the level of the alarm, the alarm reason, the treatment method. In addition, the touch screen 121 in the lock screen protection state may also display the pump information and the lock screen identification of the infusion pump 100.

In one example, an operating user (a medical worker, a staff member, etc.) may carry out manipulation commands such as setting, adjusting, stopping of the infusion pump 100 using the touch screen 121.

In the first screen control mode provided by the embodiments of this invention, the processing of the manipulation commands received by the touch screen 121 can be stopped when the pump door 12 of the infusion pump 100 is opened, which can be understood as controlling the touch screen 121 of the infusion pump 100 to enter a lock screen protection state, so as to prevent incorrect operation or interference with the normal operation of the infusion pump 100.

In the second screen control mode, the processing of the manipulation commands received by the touch screen 121 can be stopped in response to the closing operation of the pump door 12 of the infusion pump 100 to prevent incorrect operation or interference with the normal operation of the infusion pump 100. Further, the processing of the manipulation commands received by the touch screen 121 may be resumed in response to the received unlocking command, thereby improving the safety and reliability of the infusion pump 100.

Further, the embodiment of this invention may resume processing of the manipulation commands received by the touch screen 121 according to the received unlocking command, thereby improving the safety and reliability of the infusion pump 100.

In one example, the embodiment of this invention may also receive the unlocking command by means of the touch screen 121 or a physical button. In addition, corresponding to the first and second screen control modes, respectively, the unlocking command may also be the opening or closing operation of the pump door 12 of the infusion pump 100. When the pump door 12 of the infusion pump 100 being closed or opened is detected, the processing of the manipulation command received by the touch screen 121 is resumed.

In an embodiment, for example, when the operating user needs to replace the infusion bag or clean the infusion tube, the pump door 12 of the infusion pump 100 needs to be opened. After the pump door 12 is opened, the touch screen 121 is automatically locked and no longer responds to any manipulation commands, so that the operating user can avoid mistakenly touching or sliding on the touch screen 121, which results in the parameters of the infusion pump 100 being changed or stopping the work. When the operating user needs to close the pump door 12 of the infusion pump 100 after completing the replacement of the infusion bag or the cleaning of the infusion tube, the touch screen 121 may display an unlocking button, and the operating user may be required to press the button in order to unlock the touch screen 121. Or a key input interface may be displayed on the touch screen 121, and the operating user may be required to input the unlocking key to control the touch screen 121 to enter the unlocking display state to re-receive the manipulation command of the operating user. Of course, it is also possible to resume processing of the manipulation command received by the touch screen 121 in response to a closing operation of the pump door 12 of the infusion pump 100. For specific realization, settings may be made configured according to practical needs.

In this way, when the operating user installs or replaces the consumable, or starts or exits the infusion, inadvertent touching of the touch screen 121 will not affect the parameters displayed, so there will be no need for the operating user to reset the parameters modify the infusion operation. Therefore, the efficiency of operation using the infusion pump 100 will not be affected, and medical errors will not be caused as a result, and the effect of employing the infusion pump 100 to perform infusion treatment on the subject can be improved.

In the embodiments of this invention, the first screen control mode and the second screen control mode can be switched according to different operating user needs and the use of the infusion pump 100, or the operating user can manually switch, which improves the operational flexibility of the infusion pump 100 and the operating user experience.

By providing a switching method between the two screen control modes of the infusion pump 100, it is possible for the operating user to manually switch or automatically switch between the first screen control mode and the second screen control mode according to the operating user's usage. This can satisfy the different preferences and habits of different operating users and improve the intelligence and ergonomics of the infusion pump 100.

For example, the operating user may manually switch between the first screen control mode and the second screen control mode by pressing a switching button on the touch screen 121.

For another example, the infusion pump 100 may automatically switch between the first screen control mode and the second screen control mode according to data such as the frequency of use, the time of use, and the scene of use of the touch screen 121 by the operating user.

As an embodiment, if it is detected that the frequency of use of the touch screen 121 by the operating user is greater than a predetermined frequency, indicating that the operating user is frequently operating the touch screen 121 of the infusion pump 100, it may be automatically switched to adopt the second screen control mode. That is, the processing of the manipulation commands received by the touch screen 121 is stopped when the pump door 12 of the infusion pump 100 is closed.

As an embodiment, if the use scenario of the infusion pump 100 is detected as being in the process of installing or replacing infusion consumables, it may be automatically switched to adopt the first screen control mode. That is, the processing of the manipulation commands received by the touch screen 121 is stopped when the pump door 12 of the infusion pump 100 is open.

As an embodiment, if it is detected that the infusion pump 100 has been in the power-on state, and the operating user has operated the touch screen 121 for a long period of time, such as, up to half an hour, it may be automatically switched to adopt the first screen control mode. That is, when the pump door 12 of the infusion pump 100 is being opened, the processing of the manipulation commands received by the touch screen 121 is stopped.

As another embodiment, if it is detected that the infusion pump 100 has been in the “on” state, and the operating user has operated the touch screen 121 for a shorter period of time, such as, less than 5 s, the second screen control mode may be automatically switched to adopt the second screen control mode. That is, when the pump door 12 of the infusion pump 100 is being closed, the processing of the manipulation commands received by the touch screen 121 is stopped.

It is to be noted that, in the embodiments of this invention, the manner of switching between the first screen control mode and the second screen control mode is not specifically limited, but may be set and adjusted according to the practical situation.

Further, for the two control modes of the touch screen 121 of the infusion pump 100 after the door is closed, for example, the touch screen 121 is automatically unlocked after the door is closed, or the screen is kept locked after the door is closed. In the infusion scenario, the specific control method using the touch screen 121 may be according to the needs of the operating user, the use of the infusion pump 100, or the configuration of the infusion pump 100, or it may be selected based on the safety level or the operation status of the infusion pump 100 (the fault status of infusion pump 100, the status of the infusion drug, etc.), and both of the two control methods of touch screen 121 may be realized in the embodiments of this invention.

For example, for a situation where a fault is resolved such as an air bubble or a tube falling off in the infusion pump 100, the touch screen 121 of the infusion pump 100 may be controlled to remain locked, i.e., the touch screen 121 can continue to be kept locked after the pump door of the infusion pump 100 is closed, and then be unlocked by the operating user when the operating user determines that he or she wants to start the touch screen 121 of the infusion pump 100.

For another example, for some controlled medications, it is also possible to keep the touch screen 121 of the infusion pump 100 continuously locked even after the pump door of the infusion pump 100 is closed to enhance the safety of the operating user in employing the infusion pump 100 to perform the infusion operation.

For another example, for consumables installation and replacement of consumables, this can be processed in combination with operating the pump. If the infusion parameters have been set, the touch screen 121 can be kept locked after the pump door of the infusion pump 100 is closed, and then the touch screen 121 can be unlocked by the operating user when the operating user determines that the control parameters are to be adjusted. If the infusion parameters have not been set or need to be modified, the touch screen 121 can be unlocked after the pump door of the infusion pump 100 is closed, facilitating the manipulation of the infusion pump 100 by the operating user.

In an embodiment, the infusion pump 100 further includes a physical button, which is provided on the main body housing 11 and connected to the controller. The controller is further configured to stop processing of the manipulation commands received by the physical button while stopping processing of the manipulation command received by the touch screen 121; and/or resuming processing of the manipulation command received by the physical button in response to an unlocking commands received by the touch screen 121.

In the infusion pump 100 provided by the embodiment of this invention, the physical button is provided on the main body housing 11 of the infusion pump 100 may be a supplement or backup to the touch screen 121, thereby improving the operational convenience and reliability of the infusion pump 100. For example, the physical button may include a start/stop button, a speed adjustment button, and an alarm elimination buttons. The operating user may press or rotate these buttons to perform manipulation commands on the infusion pump 100 without the need to use the touch screen 121.

The embodiments of this invention can also implement control synchronized with the touch screen 121 on the physical button, capable of stopping the processing of the manipulation commands received by the touch screen 121 and the physical button at the same time when the pump door 12 of the infusion pump 100 is opened, in order to prevent incorrect operation or interference with the normal operation of the infusion pump 100. After the pump door 12 of the infusion pump 100 is closed, the unlock command received by the touch screen 121 may be used to resume the processing of the manipulation commands received by the touch screen 121 and the physical button simultaneously.

For example, when the operating user needs to replace the infusion bag or clean the infusion tube, the pump door 12 of the infusion pump 100 needs to be opened, and at this time, the touch screen 121 and the physical button may automatically lock and no longer respond to any manipulation commands, so as to prevent the operating user from mistakenly touching or pressing on the touch screen 121 or the physical button which results in the parameter of the infusion pump 100 being changed or stopping the work. When the operating user needs to close the pump door 12 of the infusion pump 100 after completing the replacement of the infusion bag or the cleaning of the infusion tube, the touch screen 121 may display an unlocking button, which the operating user needs to press in order to unlock the touch screen 121 and the physical button, and to revert to the manipulation commands.

In addition, when the operating user performs unlocks the touch screen 121, the physical buttons may also synchronize to give corresponding feedback signals, such as sound or light, in order to prompt the operating user that the unlocking has been successful.

In an embodiment, the controller triggers the locking operation of the touch screen 121 according to the door opening state, which may be as follows: triggering the stopping of processing of the manipulation command received by the touch screen 121 according to a set locking condition.

The locking condition may be adjusted according to the unlocking operation data of the operating user of the infusion pump 100 performing the unlocking operation.

In an embodiment, the above locking condition may, for example, be any of the following.

First locking condition—trigger according to the opening status of the pump door, including at least one of the following: trigger the stopping as soon as the pump door 12 is opened, detecting that the angle/degree of opening of the pump door 12 reaches a preset condition, or that the pump door 12 has finished opening.

Second locking condition-triggered by the pump door 12 detecting device detecting after the pump door 12 has been open for a preset length of time.

The embodiments of this invention may also adjust the locking conditions according to different user needs and/or use of the infusion pump 100. According to unlocking operation data of the unlocking operation performed by the operating user, it is selected to stop immediately or to wait for a preset length of time before stopping processing of the manipulation commands received by the touch screen 121, which improves the flexibility of the operation of the infusion pump 100 and the experience of the operating user.

The embodiments of this invention may provide two or more locking conditions for the operating user to select, for example, the operating user may select to trigger the locking of the touch screen 121 as soon as the pump door 12 is detected as having been opened, or wait for the pump door 12 to be opened for a preset length of time before triggering the locking of the touch screen 121 and stopping the processing of the manipulation commands received by the touch screen 121. In an embodiment, the operating user may select, or modify, the locking conditions, including setting or modifying the preset time duration by means of a corresponding control on the touch screen 121.

Further, the locking conditions may be determined or adjusted according to the unlocking operation data of the unlocking operation performed by the operating user to adapt to the operating habits and needs of different operating users, which improves the intelligence and ergonomics of the infusion pump 100. The unlocking operation data characterizes the operating user's execution of the unlocking operation and/or the related processing after the execution of the unlocking operation, and may include one or more of a number of times of the unlocking operation, a frequency of the unlocking operation, a duration of the locking screen before the unlocking, an operation after the unlocking, and the like.

In an embodiment, the infusion pump 100 may determine or adjust the locking conditions according to data such as the number of times the operating user performs the unlocking operation, the duration of the locking screen before unlocking, the frequency of the unlocking operation, and the operation after unlocking. If the operating user often performs the locking operation immediately or within a short time after opening the pump door 12 to trigger the locking screen of the touch screen 121, the preset time length for triggering the locking screen may be appropriately lengthened to minimize the inconvenience of frequent unlocking required by the operating user. If the operating user often performs the locking operation relatively long after opening the pump door 12 to trigger the locking screen of the touch screen 121, the preset time length may be appropriately shortened. Alternatively, the locking conditions may be adjusted according to the operation of the operating user after unlocking. In an embodiment, according to the operation performed by the operating user after unlocking associated with the state or mode before the pump door 12 is opened, if the state or mode before the pump door 12 is opened is associated with the operation frequently being performed by the operating user after unlocking, this state or mode before the pump door 12 is opened may be excluded from the locking condition to avoid that the operating user may need to unlock again after the pump door 12 is opened triggering the locking screen of the touch screen 121, causing unnecessary locking and unlocking operations on the touch screen 121.

In addition, the infusion pump 100 may also be set with one locking condition, or with at least two different locking conditions. For example, the locking condition may include unlocking after the pump door 12 is closed, unlocking after the pump door 12 is closed for a period of time, or unlocking after the operating user enters an unlocking password or an unlocking gesture, or unlocking after the unlocking command is entered by a mechanical button. Further, the locking conditions may also be adjusted according to the unlocking operation data of the unlocking operation performed by the operating user, including one or more of changing the locking conditions, adjusting the locking time, and adjusting the unlocking password or the unlocking gesture, and the like, so as to adapt to the operating habits and needs of different operating users, and to improve the intelligence and ergonomics of the infusion pump 100.

In another embodiment, the infusion pump 100 may set different user privileges and unlocking keys for different levels of operating users, and a corresponding information display interface may be displayed under different levels of privileges. Moreover, different operating users have different user privileges, and there is a difference in the range of privileges for the operations that can be performed on the touch screen 121 of the same infusion pump 100. Therefore, by displaying the information display interface corresponding to the user privileges of the operating user on the touch screen 121, the operating user without privileges can be avoided from performing parameter setting or modification operations on the infusion pump 100.

In an embodiment, the locking time of the touch screen may also be adaptively adjusted according to the usage habits of the operating user. When the operating user frequently unlocks the touch screen, the length of time for the touch screen to enter the lock screen state may be automatically controlled to adaptively increase.

Embodiments of this invention further provide a screen control method for an infusion pump, which can be applied to or operated on the infusion pump 100 shown in FIG. 1. FIG. 2 is a flowchart of a screen control method for an infusion pump provided by embodiments of this invention. As shown in FIG. 2, the method includes the following steps.

S201, an information display interface is generated according to the operating state of the infusion pump.

S202, the information display interface is displayed by a touch screen of the infusion pump.

S203, in response to an opening operation of the pump door of the infusion pump, the processing of a manipulation command received by the touch screen is stopped.

Specific application scenarios of this invention may be, but are not limited to, clinical medical or healthcare scenarios in which an infusion pump is used. The infusion pump is preferably an intelligent injection device that pushes out the liquid in the infusion consumable connected to the infusion pump by a drive device, and is capable of accurately, uniformly, and continuously inputting medication into the body of the patient, in order to realize the infusion treatment of the patient.

Using the infusion pump 100 not only allows for strict control of medication dosage, ensuring the optimal effective concentration of the medication, and reasonably adjusting the injection speed, but also enables the continuous infusion of various urgently needed medications, reducing the occurrence of complications. Moreover, the infusion pump is easy to operate, provides precise and reliable infusion, and can effectively reduce the workload of clinical nursing.

In the embodiments of this invention, by generating and displaying a corresponding information display interface according to the operating state of the infusion pump 100, it is convenient for the operating user to view the information display interface displayed on the touch screen 121 of the infusion pump 100, and to understand and operate the infusion pump 100. For example, when the infusion pump 100 is in a standby state, the information display interface may display basic information such as the model number, power, time of the infusion pump 100. When the infusion pump 100 is in a working state, the information display interface can display important information such as the type of fluid to be infused, the flow rate, and the remaining volume. When the infusion pump 100 is in an alarm state, the information display interface can display prompt information such as the level of the alarm, the alarm reason, the treatment method.

In one example, the operating user can also perform manipulation commands such as setting, adjusting, and stopping of the infusion pump 100 by using the touch screen 121.

In one example, an embodiment of this invention automatically stops processing of the manipulation commands received by the touch screen 121 when it monitors that the pump door of the infusion pump 100 is opened, so as to prevent the operating user from inadvertently touching the touch screen 121 and interfering with the normal operation of the infusion pump 100. For example, when the operating user needs to replace the infusion bag, clean the infusion tube, install or replace consumables, and other operations that require to open the pump door, in order to avoid mistakenly touching the touch screen 121 and changing the parameters displayed or set on the touch screen 121, it is possible to control the touch screen 121 to automatically lock and no longer respond to any manipulation commands, so as to prevent the operating user from mistakenly touching or sliding on the touch screen 121 to cause the parameters of the infusion pump 100 to change or stop working.

Further, the embodiment of this invention may resume processing of the manipulation commands received by the touch screen 121 according to the received unlocking command, which improves the safety and reliability of the infusion pump 100.

In one example, the embodiment of this invention may also receive the unlocking command by means of the touch screen 121, a physical button, and the like. Furthermore, the unlocking command may also be a closing operation of the pump door of the infusion pump 100, and the touch screen 121 may be controlled to unlock while detecting that the pump door of the infusion pump 100 is closed, in order to resume processing of the manipulation command received by the touch screen 121.

By resuming processing of the manipulation command received by the touch screen 121 in response to the unlocking command received by the touch screen 121, the safety and reliability of the infusion pump 100 are improved. For example, when the operating user needs to close the pump door of the infusion pump 100 after completing the replacement of the infusion bag or the cleaning of the infusion tube, after monitoring that the pump door of the infusion pump 100 is closed, the touch screen 121 displays an unlocking button, which the operating user needs to press in order to unlock the touch screen 121 to resume processing of the manipulation commands. For another example, after monitoring that the pump door of the infusion pump 100 is closed, the touch screen 121 may also display an unlocking interface, in which the operating user needs to enter an unlocking password (a number, a letter, a symbol, or a combination of the above elements) in order to unlock the touch screen 121, and carry out the manipulation commands again. This can prevent the normal operation of the infusion pump 100 from being affected by mistaken unlocking or malicious unlocking by others.

Of course, in the embodiment of this invention, it is also possible to resume processing the above touch screen 121 to receive the manipulation command in response to the closing operation of the pump door of the infusion pump 100. The specific realization may be set according to the practical needs.

In the control method for the touch screen 121 provided by the embodiments of this invention, when the operating user installs or replaces the, or starts or exits the infusion, it can be realized to avoid mistakenly touching the touch screen 121 and changing the displayed or set parameters, and it is not necessary for the operating user to set or modify the the parameters again. Therefore, it will not affect the operating efficiency of using the infusion pump 100, and it will not thus cause medical accidents, and the effect of using the infusion pump 100 to perform infusion treatment on the execution subject can be enhanced.

In an embodiment, the method further includes the following step.

The processing of the manipulation command received by the touch screen is stopped in response to the closing of the pump door of the infusion pump.

In embodiments of this invention, by stopping processing of the manipulation command received by the touch screen 121 in response to the closing of the pump door of the infusion pump 100, the normal operation of the infusion pump 100 can be prevented from being operated incorrectly or interfered with. Further, the processing of the manipulation commands received by the touch screen 121 can be resumed in response to the received unlocking command, thereby improving the safety and reliability of the infusion pump 100.

The embodiments of this invention may provide two control methods for the touch screen 121 of the infusion pump 100 after the door is closed. For example, the touch screen 121 can be automatically unlocked, or the screen may be kept locked when the door is closed. Depending on the infusion scenario, the specific control method chosen for the touch screen 121 may be determined based on the needs of the operating user, the use of the infusion pump 100, or the configuration of the infusion pump 100, or may be selected based on the safety level or the operating state (malfunction, infusion, etc.) of the infusion pump 100, and both control methods for the touch screen 121 can be realized in the embodiments of this invention.

For example, for a situation where a fault is resolved, such as an air bubble or a tube falling off, in the infusion pump 100, the touch screen 121 of the infusion pump 100 may be controlled to remain locked, i.e., the touch screen 121 can continue to be kept locked after the pump door of the infusion pump 100 is closed, and then be unlocked by the operating user when the operating user determines that he or she wants to start the touch screen 121 of the infusion pump 100.

For another example, for some controlled medications, it is also possible to keep the touch screen 121 of the infusion pump 100 continuously locked even after the pump door of the infusion pump 100 is closed to enhance the safety of the operating user in employing the infusion pump 100 to perform the infusion operation.

For another example, for installation and replacement of the infusion consumables, these could be processed as part of the operating state of the pump. If the infusion parameters have been set, the touch screen 121 can be kept locked after the pump door of the infusion pump 100 is closed, and then the touch screen 121 can be unlocked by the operating user when the operating user determines that the control parameters are to be adjusted. If the infusion parameters have not been set or need to be modified, the touch screen 121 can be unlocked after the pump door of the infusion pump 100 is closed, facilitating the manipulation of the infusion pump 100 by the operating user.

In an embodiment, the method further includes the following steps.

The processing of the manipulation commands received by a physical button provided on the infusion pump is stopped while stopping processing of the manipulation commands received by the touch screen; and/or, the processing of the manipulation commands received by the physical buttons is resumed in response to an unlocking command received by the touch screen.

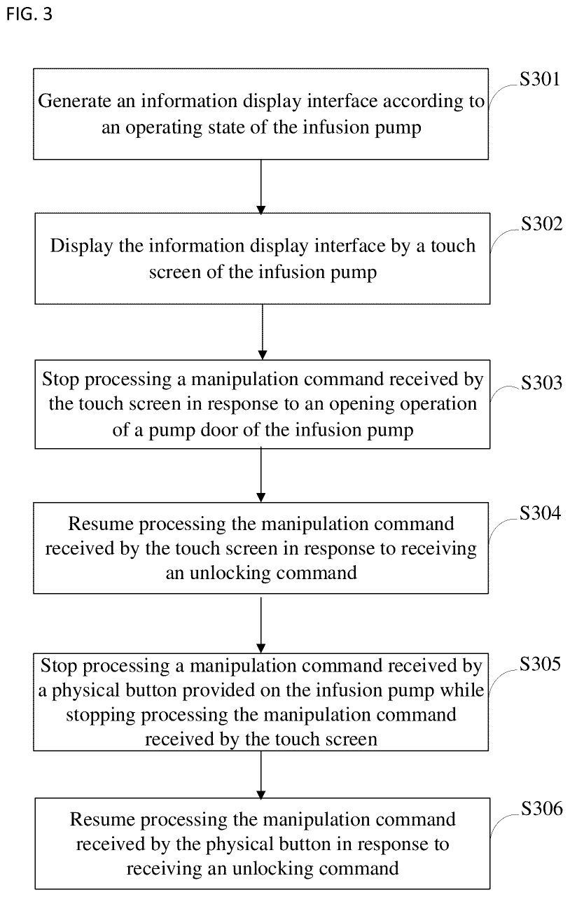

FIG. 3 is a flowchart of another screen control method for the infusion pump provided by embodiments of this invention. FIG. 3 shows an example method including both of the above method steps, and the method includes the following steps.

S301, an information display interface is generated according to an operating state of the infusion pump.

S302, the information display interface is displayed by a touch screen of the infusion pump.

S303, in response to an opening operation of the pump door of the infusion pump, the processing of a manipulation command received by the touch screen is stopped.

S304, in response to receiving an unlocking command, the processing of the manipulation command received by the touch screen is resumed.

S305, while stopping processing of the manipulation command received by the touch screen, the processing of a manipulation command received by a physical button provided on the infusion pump is stopped.

S306, in response to an unlocking command received by the touch screen, the processing of the manipulation command received by the physical button is resumed.

In the infusion pump 100 provided by the embodiment of this invention, the physical button is provided on the main body housing 11 of the infusion pump 100 as a supplement or backup to the touch screen, thereby improving the operational convenience and reliability of the infusion pump 100. For example, the physical button may include a start/stop button, a speed adjustment button, and an alarm elimination buttons. The operating user may press or rotate these buttons to perform manipulation commands on the infusion pump 100 without the need to operate by the touch screen.

The embodiments of this invention can also implement control synchronized with the touch screen 121 and the physical button, capable of stopping the processing of the manipulation commands received by the touch screen 121 and the physical button at the same time when the pump door 12 of the infusion pump 100 is opened, in order to prevent incorrect operation or interference with the normal operation of the infusion pump 100. After the pump door 12 of the infusion pump 100 is closed, the unlock command received by the touch screen 121 may be used to resume the processing of the manipulation commands received by the touch screen 121 and the physical button simultaneously, thereby improving the safety and reliability of the infusion pump 100.

For example, when the operating user needs to replace the infusion bag or clean the infusion tube, the pump door 12 of the infusion pump 100 needs to be opened, and at this time, the touch screen 121 and the physical button may automatically lock and no longer respond to any manipulation commands, so as to prevent the operating user from mistakenly touching or pressing on the touch screen 121 or the physical button, resulting in the parameter of the infusion pump 100 being changed or causing the infusion to stop. When the operating user needs to close the pump door 12 of the infusion pump 100 after completing the replacement of the infusion bag or the cleaning of the infusion tube, the touch screen 121 may display an unlocking button, which the operating user may have to press in order to unlock the touch screen 121 and the physical button, and to revert to the manipulation commands.

In addition, when the operating user unlocks the touch screen 121, the physical buttons may also synchronize to give corresponding feedback signals, such as sound or light, in order to prompt the operating user that the unlocking has been successful.

In an embodiment, the controller triggers the locking operation of the touch screen 121 according to the door opening state, which may be as follows: triggering the stopping of processing of the manipulation command received by the touch screen 121 according to a set locking condition.

The embodiments of this invention may also be configured to stop immediately or wait for a preset length of time before stopping processing of the manipulation command received by the touch screen according to different operating user needs and the use of the infusion pump 100 (the unlocking operation data of the unlocking operation performed by the operating user), which improves the operational flexibility of the infusion pump 100 and the operating user experience.

In embodiments of this invention, the locking conditions may be determined according to the data of the unlocking operation performed by the operating user. Alternatively, the locking conditions may be adjusted according to the operation of the operating user after the unlocking operation, which in turn may be adapted to the operating habits and needs of different operating users, thereby improving the intelligibility and ergonomics of the infusion pump 100.

In an embodiment of this invention, the above locking conditions may be any of the following.

First locking condition—trigger according to the opening status of the pump door, including at least one of the following: trigger the stopping as soon as the pump door 12 is opened, detecting that the angle/degree of opening of the pump door 12 reaches a preset condition, or that the pump door 12 has finished opening.

Second locking condition—triggered by the pump door 12 detecting device detecting after the pump door 12 has been open for a preset length of time.

The embodiments of this invention may also adjust the locking conditions according to different user needs and/or use of the infusion pump 100. According to unlocking operation data of the unlocking operation performed by the operating user, it is selected to stop immediately or to wait for a preset length of time before stopping processing of the manipulation commands received by the touch screen, which improves the flexibility of the operation of the infusion pump 100 and the experience of the operating user.

Embodiments of this invention may provide two or more locking conditions for the operating user to select, for example, the operating user may select to trigger the locking of the touch screen as soon as the pump door is detected to be opened, or wait for the pump door to be opened for a preset length of time before triggering the locking of the touch screen and stopping the processing of the manipulation commands received by the touch screen. In an embodiment, the operating user may select, or modify, the locking conditions, including setting or modifying the preset time duration by means of a corresponding control on the touch screen.

Further, the locking conditions may be determined or adjusted according to the unlocking operation data of the unlocking operation performed by the operating user to adapt to the operating habits and needs of different operating users, which improves the intelligence and ergonomics of the infusion pump 100. The unlocking operation data characterizes the operating user's execution of the unlocking operation and/or the related processing after the execution of the unlocking operation, and may include one or more of a number of times of the unlocking operation, a frequency of the unlocking operation, a duration of the locking screen before the unlocking, an operation after the unlocking, and the like.

In an embodiment, the infusion pump 100 may determine or adjust the locking conditions according to data such as the number of times the operating user performs the unlocking operation, the duration of the locking screen before unlocking, the frequency of the unlocking operation, and the operation after unlocking. If the operating user often performs the locking operation immediately or within a short time after opening the pump door 12 to trigger the locking screen of the touch screen, the preset time length for triggering the locking screen may be appropriately lengthened to minimize the inconvenience of frequent unlocking required by the operating user. If the operating user often performs the locking operation relatively long after opening the pump door 12 to trigger the locking screen of the touch screen, the preset time length may be appropriately shortened. Alternatively, the locking conditions may be adjusted according to the operation of the operating user after unlocking. In an embodiment, according to the operation performed by the operating user after unlocking associated with the state or mode before the pump door 12 is opened, if the state or mode before the pump door 12 is opened is associated with the operation frequently performed by the operating user after unlocking, this state or mode before the pump door 12 is opened may be excluded from the locking condition to avoid that the operating user may need to unlock again after the pump door 12 is opened triggering the locking screen of the touch screen, which causing unnecessary locking and unlocking operations on the touch screen.

In addition, the infusion pump 100 may also be set with one locking condition, or with at least two different locking conditions. For example, the locking condition may include unlocking after the pump door 12 is closed, unlocking after the pump door 12 is closed for a period of time, or unlocking after the operating user enters an unlocking password or an unlocking gesture, or unlocking after the unlocking command is entered by a mechanical button. Further, the locking conditions may also be adjusted according to the unlocking operation data of the unlocking operation performed by the operating user, including one or more of changing the locking conditions, adjusting the locking time, and adjusting the unlocking password or the unlocking gesture, and the like, so as to adapt to the operating habits and needs of different operating users, and to improve the intelligence and ergonomics of the infusion pump 100.

In another example, when the pump door of the infusion pump 100 is opened, the processing of the manipulation commands received by the touch screen 121 may be stopped after waiting for a preset length of time to provide a certain buffer time and operating time. For example, when the operating user needs to replace the infusion bag or clean the infusion tube, the pump door 12 of the infusion pump 100 needs to be opened. In this circumstance, the touch screen will wait for a preset length of time (e.g., 5 seconds) before locking, and will no longer respond to any manipulation commands, which can give the operating user a certain amount of time to complete the operation or cancel the operation, and improves the case of operation and flexibility of the infusion pump 100.

The two optional stop-processing methods provided by the embodiments of this invention allow the operating user to choose to stop immediately or to wait for a preset length of time before stopping processing of the manipulation commands received by the touch screen. For example, the operating user may stop processing of the manipulation commands received by the touch screen immediately by pressing a stop button on the touch screen; or the operating user may stop processing of the manipulation commands received by the touch screen after waiting for a preset length of time by pressing a delay button on the touch screen.

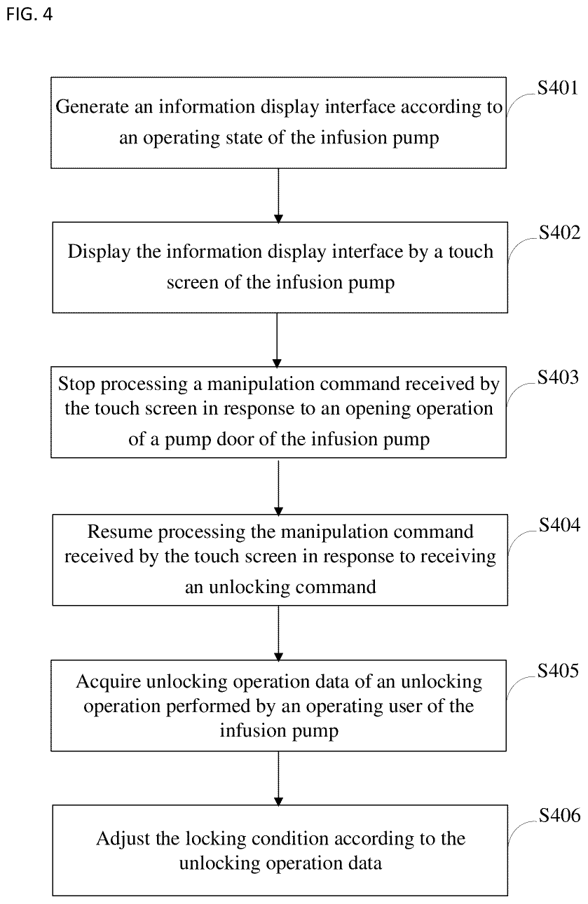

In an embodiment, FIG. 4 is a flowchart of a screen control method of an infusion pump provided by an embodiment of this invention. As shown in FIG. 4, the method includes the following steps.

S401, an information display interface is generated according to an operating state of the infusion pump.

S402, the information display interface is displayed by a touch screen of the infusion pump.

S403, in response to an opening operation of the pump door of the infusion pump, the processing of a manipulation command received by the touch screen is stopped.

S404, in response to receiving an unlocking command, the processing of the manipulation command received by the touch screen is resumed.

S405, unlocking operation data is obtained for the unlocking operation performed by the operating user of the infusion pump.

S406, the locking condition is adjusted according to the unlocking operation data.

In an embodiment, the unlocking operation data may characterize the processing related to the execution of the unlocking operation by the above-mentioned operating user, and may include one or more of the frequency of unlocking, the duration of the locking screen, the operation after the unlocking operation, the operation after the pump door is closed, and the like.

In embodiments of this invention, the locking conditions may be adjusted according to the data of the unlocking operation performed by the operating user of the infusion pump 100 to adapt to the operating habits and needs of different operating users, thereby improving the intelligence and ergonomics of the infusion pump 100.

For example, after the pump door 12 of the infusion pump 100 is closed, the unlocking command received by the touch screen 121 can be configured to resume processing of the manipulation command received by the touch screen 121 and obtain data on the operating user's execution of the unlocking operation, such as the number of times, time, and manner of the unlocking operation. For example, if the operating user often performs the unlocking operation immediately after opening the pump door 12, indicating that the operating user does not need to wait for the preset time period, then the preset time period can be shortened or canceled according to this data. If the operating user often waits for a certain period of time after opening the pump door 12 before performing the unlocking operation, indicating that the operating user needs to wait for the preset time period, then the preset time period can be lengthened or maintained unchanged according to this data.

In addition, the infusion pump 100 may also be set with one locking condition, or with at least two different locking conditions. For example, the locking condition may include unlock after the pump door 12 is closed, unlock after the pump door 12 is closed for a period of time, or unlock after the operating user enters an unlocking password or an unlocking gesture, or unlock after the unlocking command is entered by a mechanical button. Further, the locking conditions may also be adjusted according to the unlocking operation data of the unlocking operation performed by the operating user, including one or more of changing the locking conditions, adjusting the locking time, and adjusting the unlocking password or the unlocking gesture, and the like, so as to adapt to the operating habits and needs of different operating users, and to improve the intelligence and ergonomics of the infusion pump 100.

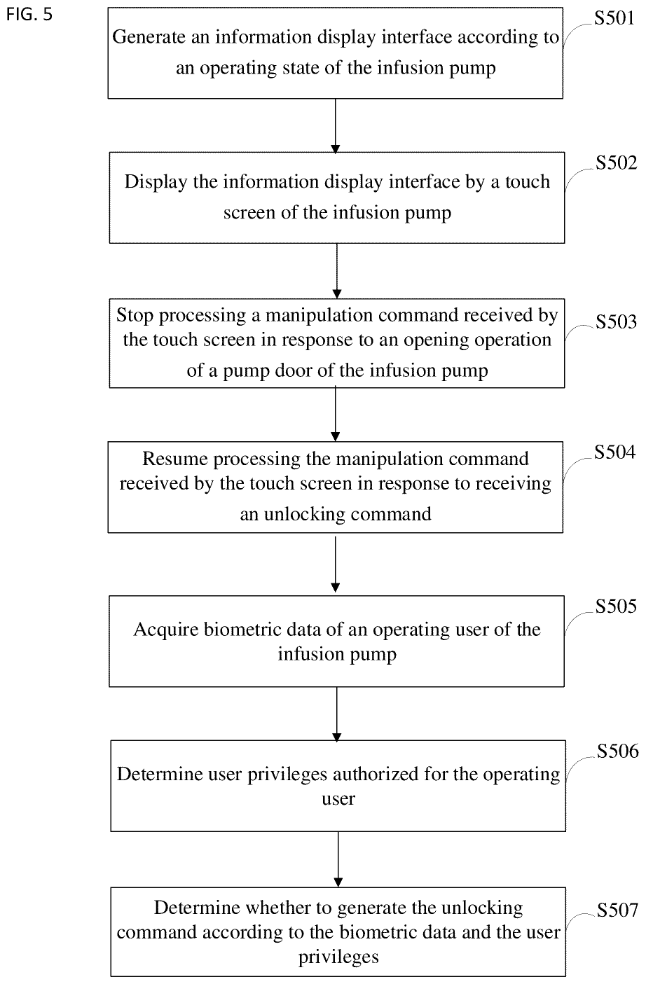

FIG. 5 is a flowchart of a screen control method of an infusion pump 100 provided by an embodiment of this invention. As shown in FIG. 5, the method includes the following steps.
S501, an information display screen is generated according to an operating state of the infusion pump.
S502, the information display interface is displayed by a touch screen of the infusion pump.
S503, in response to an opening operation of the pump door of the infusion pump, processing of a manipulation command received by the touch screen is stopped.
S504, in response to receiving an unlocking command, the processing of the manipulation command received by the touch screen is resumed.
S505, biometric data of an operating user of the infusion pump is acquired.
S506, the user privileges authorized for the operating user is determined.
S507, whether to generate the unlocking command is determined according to the biometric data and the user privilege.

In an embodiment, biometric data may be pre-recorded or pre-stored by the infusion pump 100, or may be obtained in real time from a remote server.

In one embodiment, the biometric data, such as fingerprints, iris, and voiceprints of the operating user of the infusion pump 100 is used to determine user privileges, such as an administrator, a doctor, or a nurse authorized to the operating user. According to the biometric data and the user privileges, it can be determined whether or not to generate the unlocking command in order to realize the security management and privilege control of the infusion pump 100.

For example, after the pump door 12 of the infusion pump 100 is closed, the unlocking command received by the touch screen 121 may be resumed to process the manipulation command received by the touch screen 121 and to obtain the biometric data of the operating user, such as fingerprints, iris, and voiceprints. For example, the operating user may provide the fingerprint data by pressing his or her finger on the touch screen 121, or provide the iris data by looking his or her eyes toward the touch screen 121; or provide the voiceprint data by uttering his or her name or number to the touch screen 121.

According to the biometric data of the operating user and the user privileges, it is determined whether to generate an unlocking command. For example, if the biometric data of the operating user matches the biometric data of the authorized operating user stored in the system, and the operating user has sufficient user privileges, such as an administrator or a doctor, the unlocking command is generated to allow the operating user to operate the infusion pump. If the biometric data of the operating user does not match the biometric data of the authorized operating user stored in the system, or the operating user does not have sufficient user privileges, such as a nurse or an patient, the unlocking command is not generated and the operating user is denied the ability to operate the infusion pump.

In embodiments of this invention, different operating users have different user privileges, and there is a difference in the range of privileges for the operations that can be performed on the touch screen of the same infusion pump. Therefore, by displaying an information display interface corresponding to the user privileges of the operating user on the touch screen, it is possible to avoid the operating user who does not have the privileges from setting or changing the infusion pump's operating parameters.

It should be noted that the user information (including, but not limited to, user device information, user personal information) and data (including, but not limited to, data used for analysis, stored data, displayed data) involved in this invention are all authorized by the user or are fully authorized by the parties. Besides, the collection, use and processing of the relevant data need to comply with the relevant laws, regulations and standards of the relevant countries and regions, and a corresponding operation portal may be provided for users to choose to authorize or refuse.

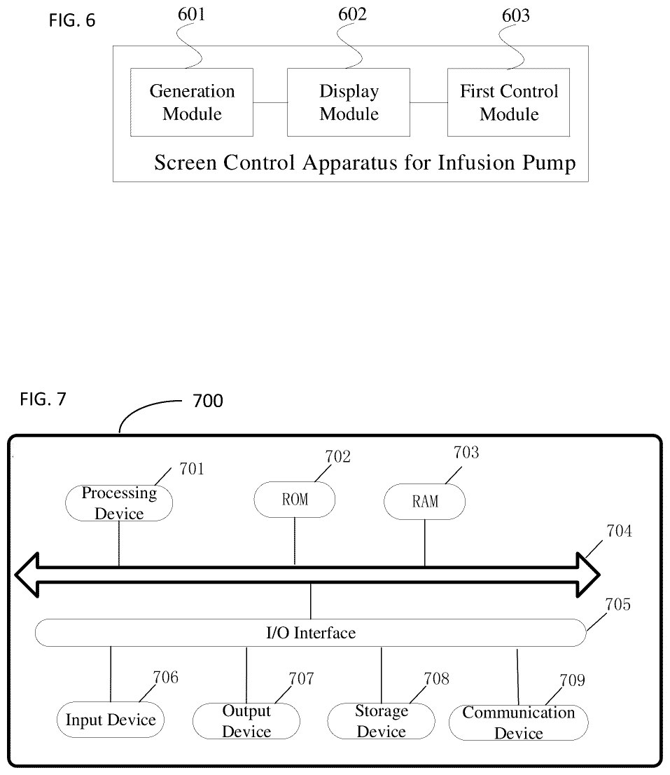

Another embodiment of this invention provides a screen control apparatus for an infusion pump. FIG. 6 shows a structural block diagram of such a screen control apparatus for an infusion pump provided by an embodiment of this invention. As shown in FIG. 6, the apparatus includes a generation module 601, a display module 602, and a first control module 603.

The generation module 601 is configured to generate an information display interface according to the operating state of the infusion pump.

The display module 602 is configured to display the information display interface by a touch screen of the infusion pump.

The first control module 603 is configured to stop processing of a manipulation command received by the touch screen in response to an opening operation of the pump door of the infusion pump.

In an embodiment, the apparatus further includes a second control module configured to resume processing of the manipulation command received by the touch screen in response to the unlocking command received by the touch screen.

In an embodiment, the apparatus further includes:
a third control module configured to stop processing of a manipulation command received by a physical button provided on the infusion pump while stopping processing of the manipulation command received by the touch screen; and
a fourth control module configured to resume processing of the manipulation command received by the physical button in response to receiving an unlocking command.

In an embodiment, the first control module is further configured to trigger the stopping of processing of the manipulation command received by the touch screen according to a set locking condition.

In an embodiment, the apparatus further includes:
a first acquisition module configured to acquire unlocking operation data of an unlocking operation performed by an operating user of the infusion pump; and
an adjustment module configured to adjust the locking condition according to the unlocking operation data.

In an embodiment, the apparatus further includes:
a second acquisition module configured to acquire biometric data of an operating user of the infusion pump;
a first determining module configured to determine user privileges authorized for the operating user; and
a second determining module configured to determine whether to generate the above unlocking command according to the above biometric data and the above user privilege.

In example embodiments, the invention further provides an electronic device including a processor, and a memory connected to the processor.

The memory stores computer-executable instructions.

The processor executes the computer-executable instructions stored by the memory to implement the method as described in for any of the embodiments disclosed above.

For example, the electronic device may be a smartphone, a tablet computer, a laptop computer, or other device that can communicate with the infusion pump, and an operating user may use the electronic device to remotely monitor and control the operating state and parameters of the infusion pump, such as viewing information such as the type, speed, and remaining volume of the infusion, or setting, adjusting, stopping, and other manipulation commands. A processor and a memory are provided in the electronic device to enable fast, accurate and stable execution of the on-screen control method of the infusion pump. For example, the processor can generate an information display interface, respond to the manipulation commands received by the touch screen, switch the screen control mode, and obtain data for the operating user to perform the unlocking operation according to the computer-executable instructions stored in the memory. The memory can store the computer-executable instructions, the operation state of the infusion pump, the biometric data of the operating user, the user privileges, and the like. In addition, the processor and the memory may exchange and synchronize data with the controller of the infusion pump to ensure consistency and real-time information between the electronic device and the infusion pump.

The embodiments of this invention further provide a computer-readable storage medium, the computer-readable storage medium having computer-executable instructions stored therein, which are configured to realize a method such as any of the foregoing when executed by a processor. For example, the computer-readable storage medium may be a CD-ROM, a hard disk, a memory card, or other data-storable medium. The operating user may load and execute the computer-executable instructions by connecting the computer-readable storage medium to an electronic device or an infusion pump to implement a method of on-screen control of the infusion pump.

In example embodiments, embodiments of this invention also provide a computer program product including a computer program. The computer program implements any one of the above-mentioned methods when executed by a processor, such as, generate an information display interface according to an operating state of the infusion pump, display the information display interface by a touch screen of the infusion pump, response to an opening operation of a pump door of the infusion pump, and stop processing of a manipulation command received by the touch. For example, the computer-executable instructions may include a series of program codes, functions, variables, parameters, and the like for defining and implementing the logic and flow of the screen control method for the infusion pump. The processor may be one or more chips or modules in the electronic device or the infusion pump for reading and executing the computer-executable instructions stored in the computer-readable storage medium, so as to realize the rapid, accurate, and stable execution of the screen control method for the infusion pump.

In order to realize the above embodiments, embodiments of this invention further provide an electronic device. FIG. 7 illustrates a structural schematic diagram of an electronic device 700 suitable for use in realizing the embodiments of this invention. The electronic device 700 may be a terminal device or a server. The terminal device may include, but is not limited to, a cell phone, a laptop computer, a digital radio receiver, a messaging device, a game console, a medical device, a fitness device, a personal digital assistant (PDA), a portable android device (PAD), a portable media player (PMP), an in-vehicle terminal (e.g., in-vehicle navigation terminal), and a fixed terminal such as a digital TV and a desktop computers. The electronic device illustrated in FIG. 7 is merely an example and should not impose any limitation on the functions and scope of use of the embodiments of this invention.

As shown in FIG. 7, the electronic device 700 may include a processing device 701 (e.g., a central processor, a graphics processor) that may execute various kinds of programs according to a program stored in a Read Only Memory (ROM) 702, or a program loaded from a storage device 708 into a Random Access Memory (RAM) 703 to perform various appropriate actions and processes. The RAM 703 also stores various programs and data necessary for the operation of the electronic device 700. The processing device 701, the ROM 702, and the RAM 703 are connected to each other through the bus 704. The input/output (I/O) interface 705 is also connected to the bus 704.

Typically, the following devices may be connected to the I/O interface 705: an input device 706 including, such as, a touch screen, a touch pad, a keyboard, a mouse, a camera, a microphone, an accelerometer, and a gyroscope; an output device 707 including, such as, a liquid crystal display (LCD), a speaker, and a vibrator; a storage device 708 including, such as, a tape and a hard disk; and a communication device 709. The communication device 709 may allow the electronic device 700 to communicate wirelessly or wiredly with other devices to exchange data. Although FIG. 7 illustrates electronic device 700 with various devices, it should be understood that it is not required to implement or have all of the illustrated devices, and that more or fewer devices may alternatively be implemented or had.

In particular, according to embodiments of this invention, the process described above with reference to the flowcharts may be implemented as a computer software program. For example, embodiments of this invention include a computer program product including a computer program carried on a computer readable medium, and the computer program includes a program code for performing the method shown in the flowchart. In such embodiments, the computer program may be downloaded and installed from a network through a communication device 709, or from a storage device 708, or from a ROM 702. When this computer program is executed by the processing device 701, the above-mentioned functions as defined in the method of an embodiment of this invention are performed.

It is noted that the computer-readable medium described above in this invention may be a computer-readable signal medium or a computer-readable storage medium or any combination of the above. The computer-readable storage medium may be, but is not limited to, an apparatus or device of electricity, magnetism, light, electromagnetism, infrared, or semiconductors, or any combination of the above. More specific examples of computer-readable storage media may include, but are not limited to: an electrical connecting device having one or more wires, a portable computer disk, a hard disk, a random access memory (RAM), a read-only memory (ROM), an erasable programmable read-only memory (EPROM or flash memory), a fiber optic, a portable compact disk read-only memory (CD-ROM), an optical storage device, a magnetic memory device, or any suitable combination of the above. In this invention, the computer-readable storage medium may be any tangible medium that contains or stores a program that may be used by or in combination with an instruction execution apparatus or device. In this invention, the computer-readable signal medium may include a data signal propagated in baseband or as part of a carrier carrying computer-readable program code. Such propagated data signals may take a variety of forms, including, but not limited to, electromagnetic signals, optical signals, or any suitable combination of the above. The computer-readable signal medium may also be any computer-readable medium other than a computer-readable storage medium, and the computer-readable signal medium can send, propagate, or transmit a program for use by, or in conjunction with, an instruction-executing device or apparatus. The program code contained on the computer-readable medium may be transmitted using any suitable medium, including, but not limited to: wire, fiber optic cable, radio frequency (RF), or any suitable combination of the above.

The computer-readable medium described above may be contained in the electronic device described above, or it may exist separately and not be assembled into the electronic device.

The computer-readable medium carries one or more programs that, when the above-mentioned one or more programs are executed by the electronic device, cause the electronic device to perform the method shown in the above-mentioned embodiments.

Computer program code for performing the operations of this invention may be written in one or more programming languages or combinations thereof, including object-oriented programming languages, such as Java, Smalltalk, C++, and conventional procedural programming languages, such as “C” language or similar programming languages. The program code may be executed entirely on the operating user's computer, partially on the operating user's computer, as a stand-alone software package, partially on the operating user's computer and partially on a remote computer, or entirely on a remote computer or server. In situations involving a remote computer, the remote computer may be connected to the operating user computer via any kind of network, including a Local Area Network (LAN) or a Wide Area Network (WAN), or may be connected to an external computer (e.g., by utilizing an Internet Service Provider to connect through the Internet).

The flowcharts and block diagrams in the accompanying drawings illustrate the architecture, functionality, and operation that may be achieved by the methods and computer program products in accordance with various embodiments of this invention. At this point, each box in the flowcharts or block diagrams may represent a module, program segment, or portion of code that contains one or more executable instructions for implementing a specified logical function. It should also be noted that in some implementations as replacements, the functions labeled in the boxes may also occur in a different order than those labeled in the accompanying drawings. For example, two consecutively represented boxes may actually be executed substantially in parallel, and they may sometimes be executed in reverse order, depending on the function involved. It is also noted that each of the boxes in the block diagram and/or flowchart, and combinations of the boxes in the block diagram and/or flowchart, may be implemented with a dedicated hardware-based system that performs the specified function or operation, or may be implemented with a combination of dedicated hardware and computer instructions.

The units described as being involved in embodiments of this invention may be implemented by way of software or may be implemented by way of hardware. For example, the first acquisition unit may be described as “a unit configured to acquire at least two Internet protocol addresses”.

The functions described above herein may be performed, at least in part, by one or more hardware logic components. For example, without limitation, example types of hardware logic components that may be used include: a field programmable gate arrays (FPGA), an application-specific integrated circuits (ASIC), an application-specific standard products (ASSP), a systems-on-chip (SOC), and a complex programmable logic devices (CPLD).

In the context of this invention, a machine-readable medium may be a tangible medium that may contain or store a program for use by or in conjunction with an instruction execution device or apparatus. The machine-readable medium may be a machine-readable signal medium or a machine-readable storage medium. The machine-readable medium may include, but is not limited to, an electronic, magnetic, optical, electromagnetic, infrared, or semiconductor device or apparatus, or any suitable combination of the above. More specific examples of machine-readable storage media would include electrical device with one or more wires, portable computer disks, hard disks, random access memory (RAM), read-only memory (ROM), erasable programmable read-only memory (EPROM or flash memory), optical fibers, convenient compact disk read-only memory (CD-ROM), optical storage devices, magnetic storage devices, or any suitable combination of the above.

Other embodiments of this invention will readily come to mind to those skilled in the art upon consideration of the specification and practice of the invention disclosed herein. This invention is intended to cover any variations, uses, or adaptations of this invention that follow the general principles of this invention and include common knowledge or customary technical means in the art not disclosed herein. The specification and embodiments are to be regarded as examples only, and the true scope and spirit of this invention is indicated by the following claims.

It is to be understood that this invention is not limited to the precise structure which has been described above and illustrated in the accompanying drawings, and that various modifications and changes may be made without departing from its scope. The scope of this invention is limited only by the appended claims.

1. A method for controlling an infusion pump, comprising:
generating an information display interface according to an operating state of the infusion pump;
displaying the information display interface on a touch screen of the infusion pump; and
stopping processing of a manipulation command received by the touch screen in response to an opening operation of a pump door of the infusion pump.

2. The method of claim 1, further comprising resuming processing of the manipulation command received by the touch screen in response to receiving an unlocking command.

3. The method of claim 1, further comprising:
stopping processing of a manipulation command received by a physical button provided on the infusion pump while stopping processing of the manipulation command received by the touch screen; and
resuming processing of the manipulation command received by the physical button in response to receiving an unlocking command.

4. The method of claim 1, further comprising resuming processing of the manipulation command received by the physical button in response to receiving an unlocking command.

5. The method of claim 1, wherein the step of stopping processing of the manipulation command received by the touch screen comprises triggering the stopping of processing of the manipulation command received by the touch screen according to a set locking condition.

6. The method of claim 5, wherein, after the step of stopping processing of the manipulation command received by the touch screen, the method further comprises:
acquiring unlocking operation data of an unlocking operation performed by an operating user of the infusion pump; and
adjusting the locking condition according to the unlocking operation data.

7. The method of claim 1, further comprising:
acquiring biometric data of an operating user of the infusion pump;
determining user privileges authorized for the operating user; and
determining whether to generate the unlocking command according to the biometric data and the user privileges.

8. An infusion pump, comprising:
a drive device,
a main body housing,
a pump door,
a touch screen,
a pump door detecting device,
a storage device, and
a controller;
wherein
the pump door is movably connected to the main body housing;
the drive device, the pump door detecting device, the storage device, and the controller are provided within the main body housing;
the touch screen is provided on a side of the pump door away from the main body housing;
the pump door detecting device is configured to detect an opening state of the pump door, and the controller is configured to control the drive device to push out the liquid in an infusion consumable connected to the infusion pump;
the controller is further configured to:
generate an information display interface according to an operating state of the infusion pump;
display the information display interface by a touch screen of the infusion pump; and
stop processing of a manipulation command received by the touch screen in response to an opening operation of the pump door of the infusion pump.

9. The infusion pump of claim 8, wherein the controller is further configured to resume processing of the manipulation command received by the touch screen in response to receiving an unlocking command.

10. The infusion pump of claim 8, further comprising
a physical button provided on the main body housing and connected to the controller; and
wherein the controller is further configured to stop processing of a manipulation command received by the physical button provided on the infusion pump while stopping processing of the manipulation command received by the touch screen and to resume processing of the manipulation command received by the physical button in response to receiving an unlocking command.

11. The infusion pump of claim 8, further comprising:
a physical button provided on the main body housing and connected to the controller;
wherein the controller is further configured to resume processing of the manipulation command received by the physical button in response to receiving an unlocking command.

12. The infusion pump of claim 8, wherein the controller is further configured to trigger the stopping of processing of the manipulation command received by the touch screen according to a set locking condition.

13. An electronic device, comprising: a processor, and a memory connected to the processor;
wherein
the memory stores computer-executable instructions;
the processor executes computer-executable instructions stored in the memory to:
generate an information display interface according to an operating state of the infusion pump;
display the information display interface by a touch screen of the infusion pump; and
stop processing of a manipulation command received by the touch screen in response to an opening operation of the pump door of the infusion pump.

14. The electronic device of claim 13, wherein the processor is further configured to resume processing of the manipulation command received by the touch screen in response to receiving an unlocking command.

15. The electronic device of claim 13, wherein the processor is further configured to stop processing of a manipulation command received by a physical button provided on the infusion pump while stopping processing of the manipulation command received by the touch screen; and
resume processing of the manipulation command received by the physical button in response to receiving an unlocking command.

16. The electronic device of claim 13, wherein the processor is further configured to resume processing of the manipulation command received by the physical button in response to receiving an unlocking command.

17. The electronic device of claim 13, wherein the processor is further configured to trigger the stopping of processing of the manipulation command received by the touch screen according to a set locking condition.

18. The electronic device of claim 17, wherein after the step of stopping processing of the manipulation command received by the touch screen, the processor is further configured to:
acquire unlocking operation data of an unlocking operation performed by an operating user of the infusion pump; and
adjust the locking condition according to the unlocking operation data.

19. The electronic device of claim 13, wherein the processor is further configured to:
acquire biometric data of an operating user of the infusion pump;
determine user privileges authorized for the operating user; and
determine whether to generate the unlocking command according to the biometric data and the user privileges.
